(19) United States
(12) Patent Application Publication
(10) Pub. No.: US 2005/0107674 A1
(43) Pub. Date: May 19, 2005

(54) DC offset cancellation techniques

(76) Inventors: Jayant Parthasarathy, Minneapolis, MN (US); Babak Ziaie, St. Paul, MN (US); Aaron D. Redish, Roseville, MN (US); Arthur G. Erdman, New Brighton, MN (US); Rahul Venkateswaran, Minneapolis, MN (US)

Correspondence Address: SHUMAKER & SIEFFERT, P. A., 8425 SEASONS PARKWAY, SUITE 105, ST. PAUL, MN, 55125, US

(21) Appl. No.: 10/953,080

(22) Filed: Sep. 29, 2004

Related U.S. Application Data
(60) Provisional application No. 60/507,307, filed on Sep. 30, 2003.

(51) Int. Cl. A61B 5/05

(52) U.S. Cl. 600/301

(57) ABSTRACT

The invention provides an electro-physiological system and various techniques for substantially canceling direct current (DC) offset while amplifying low frequency biological signals, e.g. local field potentials. In particular, the measured results of the invention show a DC offset rejection ratio of approximately greater than 100 dB. In one embodiment, the invention provides a method comprising receiving a biological signal including a frequency component and a DC (direct current) component, attenuating the frequency component of the biological signal to generate a second signal, and subtracting the second signal from the biological signal to generate a third signal.

This application claims the benefit of U.S. Provisional Application Ser. No. 60/507,307, filed Sep. 30, 2003, the entire content of which is incorporated herein by reference.

### TECHNICAL FIELD

The invention relates to electro-physiological recording systems and, more particularly, to amplifiers for biological signals.

### BACKGROUND

Electro-physiological recording systems are used to record and amplify biological signals and can be used to study and monitor neural activity. The instrumentation of such a system typically includes a reference electrode and a signal electrode to sense biological signals, an amplifier to amplify the biological signals, and a processor to convert the analog biological signals to a digital signal for processing.

One problem faced in electro-physiological systems is the presence of DC offset between the electrodes, which may be 1,000 times greater than the voltage of the biological signal to be measured. Consequently, the DC offset can cause saturation of the amplifier. Discrete systems may implement a large series capacitor in order to block the DC offset. However, such a large series capacitor is not practical in an integrated circuit (IC) because of the large chip area required by the capacitor.

### SUMMARY

In general, the invention is directed to techniques for substantially canceling or removing the DC offset from detected biological signals in an electro-physiological system. In particular, the invention is directed to techniques for substantially canceling the DC offset while amplifying low frequency signals at a high DC offset rejection ratio. Unlike conventional techniques that rely on capacitors or voltage dividers to suppress the DC offset and provide for high gain in a pass band, typically 300-6,000 Hz, the techniques described herein utilize a feed-forward amplifier that includes a filter and a subtraction unit to substantially cancel the DC offset. In addition the feed-forward amplifier may amplify low frequency biological signals, e.g. local field potentials which can have a frequency of 7 Hz, in order to improve the DC offset rejection ratio. The filter and subtraction unit may be fabricated on an integrated circuit (IC), allowing the techniques to be used for applications that require small integratable systems such as neural prosthetics.

In accordance with an embodiment of the invention, a biological signal including a frequency component and a DC component is received, and the frequency component of the biological signal is attenuated to generate a second signal comprising primarily the DC component of the biological signal. A third signal is then generated by subtracting the second signal from the biological signal. The third signal may include primarily the frequency component of the biological signal. Additionally, amplification may be performed such that the third signal comprises an amplified version of the frequency component of the biological signal. In some embodiments, the DC offset rejection ratio of the third signal relative to the biological signal may be greater than approximately 100 decibels (dB).

In one embodiment, the invention is directed to a method comprising receiving a biological signal including a frequency component and a DC (direct current) component, attenuating the frequency component of the biological signal to generate a second signal, and subtracting the second signal from the biological signal to generate a third signal.

In another embodiment, the invention is directed to an amplifier comprising a filter to receive a biological signal including a frequency component and a DC component and to attenuate the frequency component to generate a second signal and a subtraction unit to subtract the second signal from the biological signal to generate a third signal.

In another embodiment, the invention is directed towards an electro-physiological system comprising a set of electrodes to identify a set of biological signals. The system also includes an amplifier comprising a filter and a subtraction unit. The filter receives a selected biological signal including a frequency component and a DC component and attenuates the frequency component to generate a second signal. The subtraction unit subtracts the second signal from the selected biological signal to generate a third signal.

In yet another embodiment, the invention is directed to an electro-physiological system comprising a set of electrodes to identify a set of biological signals, each of the biological signals including a frequency component and a DC component and a set of amplifiers corresponding to the set of electrodes. Each amplifier of the set of amplifiers comprises a filter to receive one of the biological signals and to attenuate the frequency component of the received biological signal to generate a second signal, and a subtraction unit to subtract the second signal from the received biological signal to generate a third signal.

The invention may be capable of providing one or more advantages. For example, the invention provides devices and techniques that can be implemented in integratable systems while allowing substantial DC offset cancellation and amplification of low frequency signals at a high DC offset rejection ratio. For example, the described devices and techniques can be used in wireless neural electro-physiological circuits where integrated circuit area and packaging are important parameters, such as for clinical implants and neural headstages. Furthermore, the described techniques and devices can be used in systems where it is difficult to place a large capacitor across the input because of added parasitics to the substrate. In particular, the feed-forward amplifier of the described invention may require only a single small capacitor. As a result, the feed-forward amplifier of the described invention may be implemented using complimentary metal-oxide semiconductors (CMOS), Bipolar CMOS (BiCMOS), or other integrated components. As a result, the described devices and techniques may reduce the size of currently available electro-physiological recording systems.

Moreover, the invention incorporates a filter and a subtraction unit to facilitate an amplifier that can substantially cancel the DC offset and amplify low frequency signals with a high DC offset rejection ratio. In particular, DC offset rejection ratios greater than approximately 100 dB can be achieved, even while amplifying low frequency signals. Unlike conventional systems that amplify signals in a pass band of typically 300-6,000 Hz, the described invention may amplify low frequency signals approximately less than 7 Hz.

The invention may also allow for a reduction in the number of circuit blocks in an electro-physiological system, which can significantly reduce power consumption and heating of nearby circuitry. Heating of nearby circuitry is particularly undesirable when the invention is implanted in a patient, as such heating can result in the heating of patient tissue. Furthermore, each channel of the system may exhibit more uniform DC offset cancellation with respect to conventional systems because the amount of DC offset suppression may vary from amplifier to amplifier in conventional systems. In any event, the described invention allows for an electro-physiological system with reduced size in comparison to conventional systems.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram illustrating an exemplary electro-physiological system including a feed-forward amplifier that substantially cancels the direct current DC offset and amplifies low frequency biological signals with a high DC offset rejection ratio according to an embodiment of the invention.

FIG. 2 is a block diagram illustrating an electro-physiological system including a feed-forward amplifier that substantially cancels the direct current DC offset and amplifies low frequency biological signals with a high DC offset rejection ratio according to an alternative embodiment of the invention.

FIG. 3 is a block diagram illustrating the feed-forward amplifier of FIGS. 1 and 2.

FIG. 4 is a block diagram illustrating one example of the feed-forward amplifier of FIG. 3 in greater detail.

FIG. 5 is a circuit diagram illustrating one embodiment of the feed-forward amplifier of FIGS. 1 and 2.

FIG. 6 is another circuit diagram illustrating another embodiment of the feed-forward amplifier of FIGS. 1 and 2.

FIG. 7 is a flowchart illustrating an example method that may be implemented by the feed-forward amplifier of FIGS. 1 and 2.

FIGS. 8A-8C are graphs illustrating the frequency spectrum representation of the direct current DC offset and frequency component at various stages of the feed-forward amplifier of FIGS. 1 and 2.

FIG. 9 is a graph illustrating the simulated magnitude response of the specific feed-forward amplifier illustrated in FIG. 5.

FIG. 10 is a graph illustrating the measured magnitude response of the specific feed-forward amplifier illustrated in FIG. 5.

FIG. 11 is a graph illustrating the magnitude response of the feed-forward amplifier illustrated in FIG. 4 with amplification factor “G” equal to 1.

FIG. 12 is a graph illustrating the simulated magnitude response of the feed-forward amplifier illustrated in FIG. 6 from 0.1 μHz to 1 kHz.

FIG. 13 is a graph illustrating the measured frequency response of an exemplary feed-forward amplifier similar to that illustrated in FIG. 6 from 10 MHz to 1 kHz.

FIG. 14 is a graph illustrating the measured transient response of an exemplary feed-forward amplifier similar to that illustrated in FIG. 6 at 1 kHz.

### DETAILED DESCRIPTION

Electro-physiological recording systems are used to record and amplify biological signals and may be used to study and monitor neural activity. The instrumentation of such a system may comprise a front end that includes a reference electrode and a signal electrode to sense biological signals, an amplifier to amplify the biological signals, and a back end that includes a processor to convert the analog biological signals to a digital signal for processing. The electrodes are typically insulated microwires or micromachined silicon electrodes. In order to reduce the loading effect in high impedance microelectrodes, high impedance buffers, e.g. field effect transistors, are typically used.

The buffered signals are then amplified by the amplifier. The amplifier may include a common-mode noise subtractor to filter out common-mode noise, a filter to suppress the DC offset generated between the electrodes, and an amplifier to amplify the filtered signal. The offset rejection ratio is the ratio of the DC offset rejection to the gain in the passband and gives a measure of the offset relative to the amplitude of the signal of interest. The DC offset generated between electrodes can be 1,000 times greater than the voltage of the signal to be measured. As a result, DC offset can cause the gain stages of the amplifier to saturate. The efficacy of the amplifier stage can be measured using the offset rejection ratio.

In a multi-electrode system, the common-mode noise subtractor may be succeeded by a multiplexer. The multiplexer can be used to selectively record channels where each channel corresponds to one of the number of electrodes. Additionally, each channel in a multi-electrode recording system may have a DC offset suppressing amplifier. The selected filtered signals are then amplified and converted to a digital signal by an analog-to-digital converter. The digital signal can then be processed by digital circuitry.

Conventional discrete systems insert a large series capacitor as a filter, in order to suppress the DC offset. However, inserting a large series capacitor is not practical in applications that require integratable systems in which the amplifier on an integrated chip (IC), such as neural prosthetics, due to the large area consumed by the required capacitors. Integratable systems that can attenuate the DC offset and to provide for high gain in the pass band, which is typically 300-6,000 Hz, are highly desirable.

Generally, two neural signal pass bands are of interest to neuroscientists, 300-9,000 Hz unit activity potentials, and 1-300 Hz local field potentials. Local field potential signals provide critical information and can have frequency as low as 7 Hz. While conventional integratable techniques reject the DC offset, such techniques may also attenuate low frequencies. Thus, such conventional techniques are not suited to amplify low frequency signals such as local field potentials. Large amplifiers may be used in such systems in order to reject the DC offset and record low frequency local field potentials. However, large amplifiers are not suitable for integratable systems because of their inherent size.

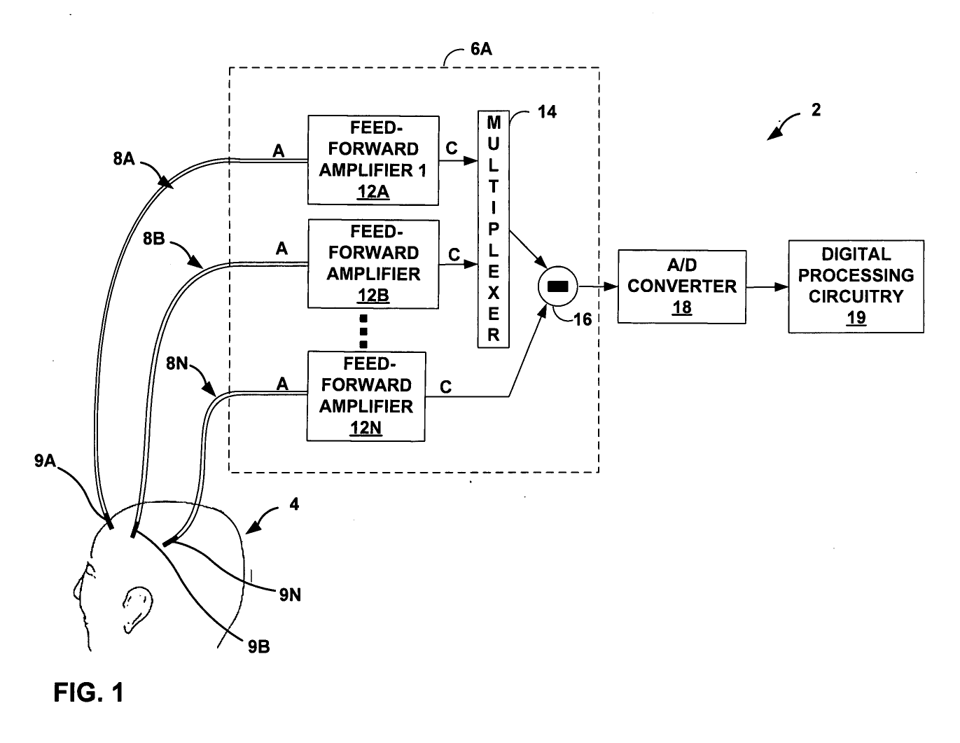

FIG. 1 is a block diagram illustrating exemplary electro-physiological system 2 that includes a set of feed-forward amplifiers 12A-12N (which are both collectively and individually referred to herein as feed-forward amplifiers 12). The feed-forward amplifiers 12 substantially cancel or remove the direct current (DC) offset and amplify low frequency biological signals of patient 4 with a high DC offset rejection ratio according to an embodiment of the invention. In general, system 2 includes a set of electrodes 9A-9N (collectively referred to herein as electrodes 9) located on leads 8A-8N (collectively referred to herein as leads 8), respectively, to identify a set of biological signals of patient 4. In particular, electrodes 9 detect voltages that represent biological signals of patient 4. Each of feed-forward amplifiers 12 receives a biological signal, labeled A in FIG. 1, generated by a corresponding one of electrodes 9 located on leads 8, and outputs a signal labeled C in FIG. 1. The biological input signal includes a frequency component and a DC component, but the output signal includes primarily the frequency component of the biological signal.

Feed-forward amplifiers 12 may be designed using field effect transistors (FETs), which avoids the need for input buffers. In any case, feed-forward amplifiers 12 substantially cancel the DC offset of the corresponding biological signal. The DC offset can have an amplitude on order of approximately 1,000 times the amplitude of the frequency component. In some embodiments, the DC offset rejection ratio of the third signal relative to the biological signal may be greater than approximately 100 decibels (dB).

Multiplexer 14 receives the output signal corresponding to each of feed-forward amplifiers 12 and selects a combination of the output signals to output to common-mode noise subtractor 16. Common-mode noise subtractor 16 filters out the common-mode noise of the selected output signals received from multiplexer 14. The output of common-mode noise subtractor 16 is primarily an amplification of the frequency component of the biological signal. Analog-to-digital converter 18 receives the output of common-mode noise subtractor 16 and converts the received signal into a digital signal that can be processed by digital circuitry 19. Amplifiers 12, multiplexer 14, and common-mode noise subtractor 16 are fabricated on integrated circuit (IC) 6A.

The configuration of leads 8 illustrated in FIG. 1 is merely exemplary. In various embodiments, IC 6A may be connected to any number of leads that extend to a variety of positions on the body of patient 4 or within the body of patient 4. Also, multiple electrodes may be disposed on any given lead. Leads 8 extend to the cranium of patient 4 in order to measure neurological brain signals. However, leads 8 may also be implanted proximate to the spinal cord of patient 4, or in other internal locations. In any case, leads 8 include electrodes 9, which system 2 uses to sense biological signals of patient 4. In particular, leads 8A, 8B each include a signal electrode while lead 8N includes a reference electrode.

In a multi-electrode system, IC 6A may be connected with any number of signal electrodes while connected to a single reference electrode. Multiple references, may also be used and possibly selected to provide an good reference signal at any given time. In the illustrated embodiment, reference electrode 9A is preferably located at a position on or within the body of patient 4 that has relatively fewer neurons than the positions at which signal electrodes 9A, 9B are located. In some embodiments, the invention described herein may allow any of electrodes 9 to be selectively used as a reference electrode. In other words, the reference electrode may be dynamically selected. In any event, electrodes 9 included in leads 8 may be insulated microwires, micromachined silicon electrodes, or any other electrode capable of detecting voltages attendant to biological signals of patient 4.

Leads 8 are coupled to feed-forward amplifiers 12, respectively. Thus, feed-forward amplifiers 12 receive the biological signal output by a corresponding one leads 8. Each of feed-forward amplifiers 12 substantially cancel the DC offset of the corresponding biological signal and may also amplify low frequency signals. In any case, feed-forward amplifies 12 have with a high DC offset rejection ratio.

One problem faced in electro-physiological systems is that of a DC offset generated between electrodes that is typically 1,000 times greater than the voltage of the signal to be measured. Consequently, the DC offset can cause the gain the amplifier, which follows the electrodes, to saturate. Conventional discrete systems insert a large series capacitor to block the DC offset. However, such a large series capacitor is not practical in systems that integrate an amplifier because of the large circuit area required by the capacitor. An integrated amplifier is desirable for many applications of electro-physiological applications requiring a small recording system, e.g. neural prosthetics.

Conventional integrated techniques attenuate the DC offset and provide for high gain in a pass band typically of approximately 300-6,000 Hz. A pass band of 300-6,000 Hz includes unit activity potentials. However, systems that implement such conventional techniques are not capable of amplifying low frequency signals such as local field potentials that have a frequency of 1-300 Hz. Local field potentials provide critical information and have a frequency as low as 7 Hz.

Feed-forward amplifiers 12 substantially cancel the DC offset generated by leads 8 and amplify low frequency signals with a high DC offset rejection ratio. As described previously, the input biological signal includes a DC component and a frequency component. The DC offset can have an amplitude on order of approximately 1,000 times the amplitude of the frequency component. In some embodiments, the DC offset rejection ratio of the third signal relative to the biological signal may be greater than approximately 100 decibels (dB).

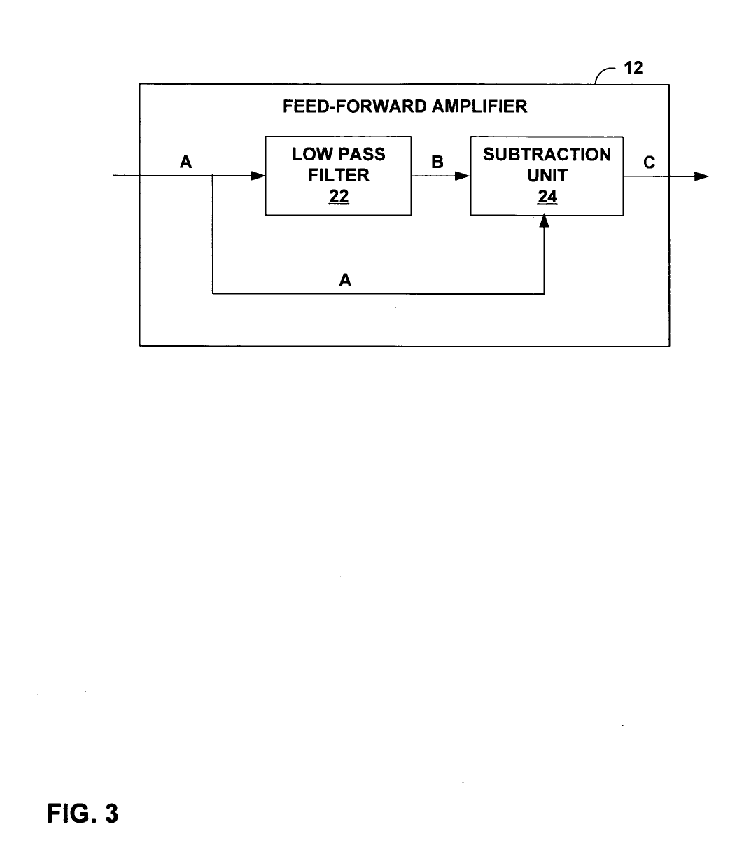

In general, the biological signal is received by one of feed-forward amplifiers 12 and passes through a first and a second path. The first and second paths are designed such that the amplitude and phase characteristics through either the first or second path are the same for the DC component but different for the frequency component of the biological signal. As will be described in greater detail with reference to FIG. 3, the first path can be realized by low pass filtering the biological signal to generate a second signal. The low pass filtering passes the DC component while attenuating the frequency component of the biological signal to generate a second signal. Consequently, the second signal includes primarily the DC component of the biological signal. The second path is a simple feed-forward path that is input to a subtraction unit. The subtraction unit also receives the second signal and, thus, subtracts the input of the low pass filter with the output of the low pass filter. More particularly, the subtraction unit subtracts the second signal from the biological signal to generate a third signal. As a result, the third signal includes primarily the frequency component of the biological signal. In other words, the low pass filter passes the DC component of the biological signal while attenuating the frequency component of the biological signal and, thus, upon subtraction of the output with the input of the low pass filter, the DC offset is substantially cancelled.

In some embodiments, the frequency component of the biological signal is less than approximately 7 Hz. Moreover, the subtraction unit may also be designed to amplify the third signal. In some embodiments, the DC offset rejection ratio of the third signal relative to the biological signal is greater than approximately 100 dB. However, the invention is not limited as such. For example, the invention described herein may be particularly advantageous in any electro-physiological recording system, including but not limited to deep brain stimulation and monitoring applications, spinal cord stimulation and monitoring applications, and other neural electro-physiological applications, or other biological sensing applications.

Multiplexer 14 receives the third signals output by feed-forward amplifiers 12 and selects a combination of the received third signals as its output. In other words, in the embodiments illustrated in FIG. 1, multiplexer 14 receives the third signal output by feed-forward amplifiers 12A, 12B and not from feed-forward amplifier 12N because feed-forward amplifier 12N corresponds to reference electrode 8N. The output of multiplexer 14 is the third signal output by one or a combination of feed-forward amplifiers 12A, 12B.

Common-mode noise subtractor 16 filters out the common-mode noise of the output of multiplexer 14 and feed-forward amplifier 12N. The output of feed-forward amplifier 12N provides common-mode noise subtractor 16 with a reference signal that can be used to substantially cancel the common-mode noise of the output multiplexer 14. Common-mode noise subtractor 16 may be a difference amplifier or any electrical component that filters out common-mode noise from electrical signals. In the preferred embodiment, amplifiers 12, multiplexer 14, and common-mode noise subtractor 16 are fabricated on IC 6A. The invention described herein may be implemented in CMOS, BiCMOS, or other technologies suitable for fabricating amplifiers 12, multiplexer 14, and common-mode noise subtractor 16 on an IC.

The back end of system 2 includes analog-to-digital converter 18 and digital circuitry 19. Analog-to-digital converter 18 converts the signal output by common-mode noise subtractor 16 into a digital signal. The digital signal can then be processed by digital circuitry 19. Analog-to-digital converter 18 and digital circuitry 19 may be discrete electrical components or possibly one or more additional integrated circuits.

As illustrated in the exemplary embodiment of FIG. 1, N leads 8 that include N electrodes 9 are coupled to N corresponding feed-forward amplifiers 12, where N represents a positive integer. In some embodiments, leads 8 may include any number of leads coupled to amplifiers 12. Also, leads 8 may include up to 50 electrodes coupled to a corresponding 50 feed-forward amplifiers 12. In another example, leads 8 may include up to 100 electrodes coupled to a corresponding 100 feed-forward amplifiers 12. Multiple electrodes may also be disposed on the leads to define additional channels. In general, the invention described herein allows multiple channels defined by a set of electrodes to be processed on an IC. In particular, the invention described herein may be particularly advantageous for applications requiring integrated systems to provide DC offset rejection and amplification of low frequency signals such as neural prosthetics, clinical implants, and neural headstages. For example, the invention described herein may be used in wireless neural electro-physiological circuits where integrated circuit area and packing are important parameters. Additionally, the invention may reduce the size of the currently available recording systems which require huge racks of amplifiers to perform equivalent tasks.

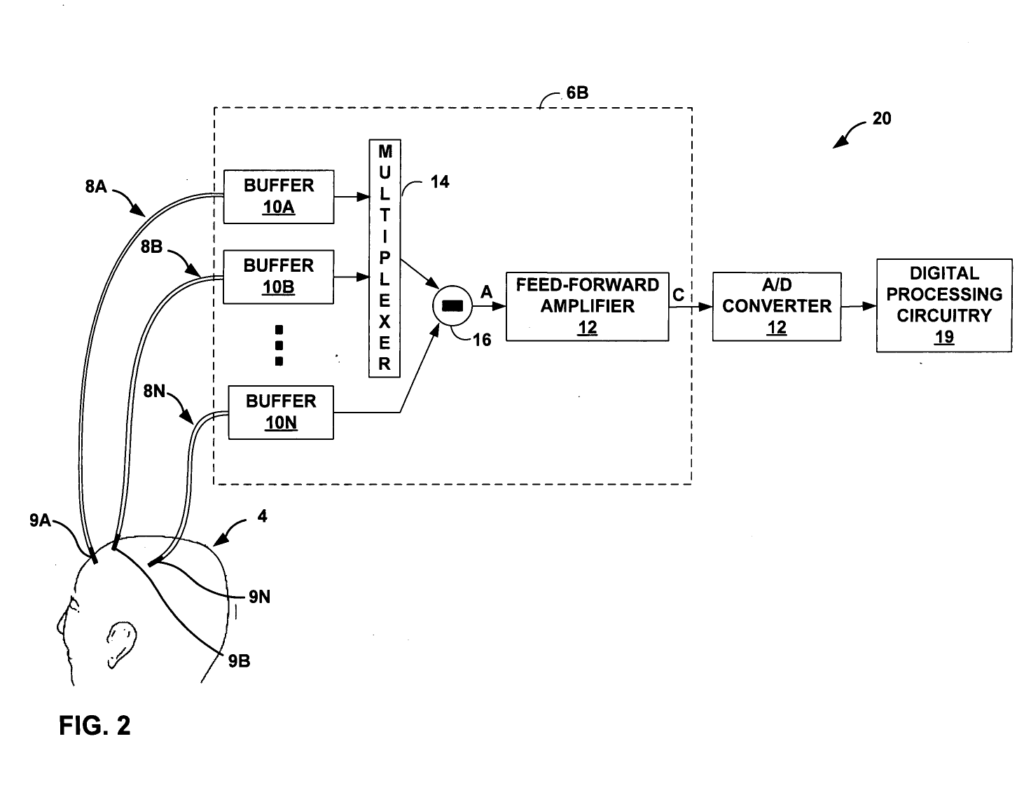

FIG. 2 is a block diagram illustrating exemplary electro-physiological system 20 that includes a single feed-forward amplifier 12 to substantially cancel the DC offset and amplify low frequency biological signals of patient 4 with a high DC offset rejection ratio according to an alternative embodiment of the invention. In general, system 20 also includes a set of electrodes 9 located on leads 8 to identify a set of biological signals of patient 4. Electrodes 9 detect voltages that represent biological signals of patient 4. Buffers 10 buffer the biological signals generated by electrodes 9 located on leads 8, respectively. Multiplexer 14 receives the output from buffers corresponding to signal electrodes and selectively outputs a combination of the buffered signals. In the illustrated embodiment of FIG. 2, for example, multiplexer 14 receives the output from buffers 10A, 10B and selectively outputs one or a combination of the received signals. Common-mode noise subtractor 16 receives the selected biological signal output by multiplexer 14 and the output of the reference electrode, i.e. the output of electrode 9A in the illustrated embodiment, and filters out the common-mode noise of the selected biological signal to generate the biological signal. The output of common-mode noise subtractor 16 is labeled A in FIG. 2 and includes a DC component and a frequency component. In the preferred embodiment of system 20, buffers 10, multiplexer 14, common-mode noise subtractor 16, and feed-forward amplifier 12 are fabricated on IC 6B. Buffers 10, multiplexer 14, common-mode noise subtractor 16, and feed-forward amplifier 12 of system 20 may be implemented in CMOS, BiCMOS, or other technologies suitable for fabricating IC 6B.

Feed-forward amplifier 12 receives the selected biological signal and outputs a signal, labeled C in FIG. 2, that includes primarily the frequency component of the biological signal. As described previously, amplifier 12 may be implemented with a low pass filter coupled to a subtraction unit, where the inputs of the subtraction unit are the input of the low pass filter and the output of the low pass filter. In general, the low pass filter passes the DC component of the biological signal while attenuating the frequency component of the biological signal in order to generate a second signal. Consequently, the second signal includes primarily the DC component of the biological signal. The subtraction unit subtracts the second signal from the biological signal to generate a third signal. Subtracting the second signal with the biological signal causes the DC component of the biological signal to be substantially cancelled, leaving only the frequency component of the biological signal. Thus, the third signal includes only the frequency component of the biological signal. In some embodiments, the frequency component of the biological signal is less than approximately 7 Hz. As described previously, the subtraction unit may also be designed to amplify the frequency component. Therefore, feed-forward amplifier 12 substantially cancels the DC offset of the selected biological signal and amplifies low frequency signals with a high DC offset rejection ratio. In some embodiments, the DC offset rejection ratio of the third signal relative to the selected biological signal may be greater than approximately 100 decibels.

The back end of system 20 includes analog-to-digital converter 18 and digital circuitry 19 and interfaces with IC 6B. Analog-to-digital converter 18 converts the output of feed-forward amplifier 12, i.e. the third signal, into a digital signal that can be processed by digital circuitry 19.

The embodiment illustrated in FIG. 2 includes a single feed-forward amplifier 12 and thus may have significantly less IC area than the illustrated embodiment in FIG. 1 as well as conventional systems. Additionally, because system 20 has a lesser number of circuit blocks than system 2, system 20 may exhibit less power consumption and heating of nearby circuitry. Heating of nearby circuitry is particularly undesirable when IC 6B is implanted patient 4, as such heating can result in the heating of the tissue of patient 4. Furthermore, variation in the amount of DC cancellation may exist between each channel of system 2 because each channel is susceptible to variation as the process and temperature vary. Such variation may result in non-uniformity. Typically DC suppressing amplifiers are designed to operate with high performance. In that case, if any of feed-forward amplifiers 12 system 2 or system 20 fail during fabrication or testing, IC 6B may need to be discarded. Consequently, system 20 may exhibit greater uniformity, less waste, and less production cost than convention systems and/or system 2.

FIG. 3 is a block diagram illustrating, in greater detail, an exemplary feed-forward amplifier 12 used in the embodiments of FIG. 1 and FIG. 2. Feed-forward amplifier 12 includes low pass filter 22 coupled to subtraction unit 24. In general, feed-forward amplifier 12 substantially cancels the DC offset of the biological signal, labeled A, and amplifies low frequency signals with a high DC offset rejection ratio.

Low pass filter 22 may comprise a first order filter with cutoff frequency substantially equal to or less than the frequency component of the biological signal. Consequently, the output of low pass filter 22, i.e. the second signal which is again labeled B, includes primarily the DC component of the biological signal. Low pass filter 22 may be implemented using various components and circuits well known in the art of analog signal processing. In some embodiments, low pass filter 22 may be implemented with a single capacitor having a capacitive value of approximately less than 0.2 pico Farads (pF). Moreover, because integrated implementation is independent of the substrate, feed-forward amplifier 12 may be implemented in CMOS, BiCMOS, or other suitable technologies capable of fabricating feed-forward amplifier 12 on an IC. The low frequency pole of low pass filter 22 may be set by the impedance at the input of feed-forward amplifier 22 and the capacitance of the capacitor of low pass filter 22.

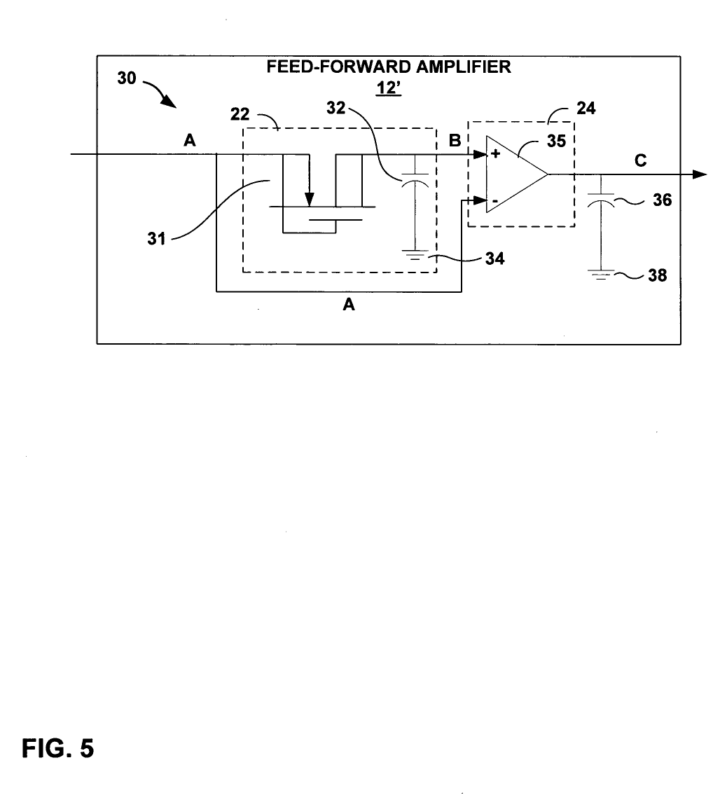

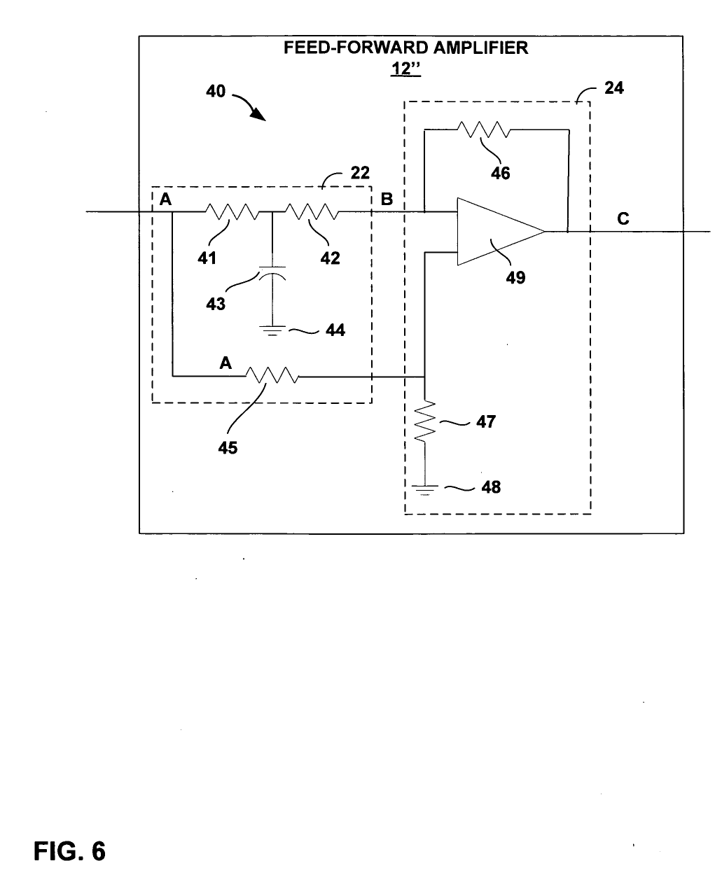

Subtraction unit 24 is generally implemented as an operational amplifier capable of amplifying the difference between its inputs. The second signal and the biological signal are received as inputs to subtraction unit 24. Subtraction unit 24 subtracts the second signal from the biological signal to generate the third signal, again labeled C, that includes primarily the frequency component of the biological signal. Subtraction unit 24 may also be implemented using various other components and circuits well known in the art. Moreover, low pass filter 22 may attenuate the frequency component to generate the second signal defined such that when the second signal is subtracted from the biological signal by subtraction unit 24 to generate the third signal, the frequency component becomes amplified in the third signal. FIGS. 5 and 6 illustrate circuit diagrams for two different implementations of exemplary feed-forward amplifiers 12′ and 12″, either of which may correspond to feed-forward amplifier 12.

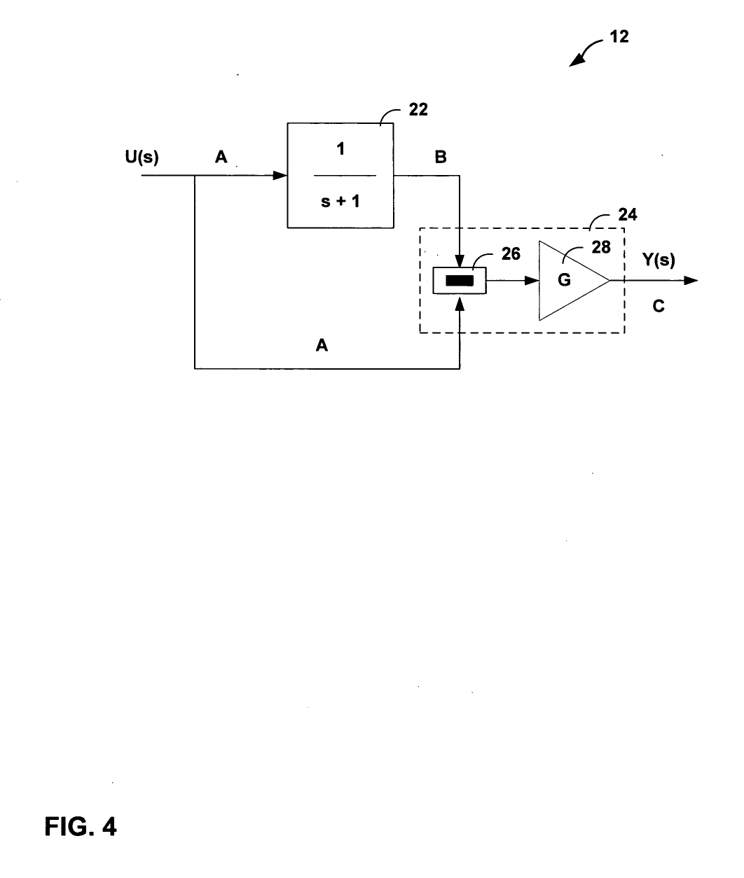

FIG. 4 is a block diagram illustrating a more detailed embodiment of feed-forward amplifier 12. As described previously, feed-forward amplifier 12 provides a first and a second path for the biological signal. The first path is realized by low pass filter 22 while the second path feeds the biological signal forward to subtraction unit 24, in accordance with the name of feed-forward amplifier 12. Low pass filter 22 passes the DC component of the biological signal while attenuating the frequency component of the biological signal. Consequently, the second signal generated as the output of low pass filter 22 includes primarily the DC component of the biological signal. Subtraction unit 24 receives the second signal and the biological signal as inputs and outputs the third signal. Subtraction unit 24 is illustrated as having a subtraction stage 26 and an amplification stage 28. Subtraction stage 26 subtracts the biological signal from the second signal to generate the third signal. Consequently, the third signal includes primarily the frequency component of the biological signal and amplification stage 24 amplifies the frequency component. In some embodiments, the frequency component of the biological signal is less than approximately 7 Hz and the ratio of the third signal relative to the biological signal is greater than approximately 100 dB.

The transfer function that describes feed-forward amplifier 12 in accordance with low pass filter 22 and subtraction unit 24 is given as equation (1) where s is the frequency component of the biological signal, U(s) is the input of amplifier 12 Y(s) is output of amplifier 12, and G is the gain of subtraction unit 24.

$$H ( s ) = \frac{Y ( s )}{U ( s )} = \frac{- Gs}{s + 1} \qquad ( 1 )$$

As expected, equation (1) is dependent on the frequency component of the biological signal. It is important to note the negative sign in equation (1) which is a result of the manner in which subtraction unit 24 subtracts its inputs.

FIG. 5 is a circuit diagram illustrating low pass filter 22 and subtraction unit 24 of an exemplary feed-forward amplifier 12′ implemented as circuit 30 that includes reverse-biased positive metal-oxide semiconductor (PMOS) transistor 31 coupled to capacitor 32 and a low noise complimentary metal-oxide semiconductor (CMOS) operational transconductance amplifier (OTA) 35, respectively. In some embodiments, capacitor 32 may have a capacitive value approximately equal to or less than 0.2 pF. Importantly, the simulated gain of circuit 30 is 44 dB with a bandwidth of 0.01-6,000 Hz. In some embodiments, the ratio of the third signal relative to the biological signal is greater than approximately 100 dB.

In this case, the combination of capacitor 32 and the impedance of reverse-biased PMOS transistor 31 set the low-frequency pole of low pass filter 22. Capacitor 32 is coupled to local ground 34. The upper limit on the bandwidth of feed-forward amplifier 12′ is set primarily by the value of load capacitor 36 seen by OTA 35 and the output resistance at the output of feed-forward amplifier 12′. Load capacitor 36 is coupled to local ground 38. External resistors are not required to set the gain. Consequently, the noise performance of feed-forward amplifier 12′ may be improved over conventional amplifiers.

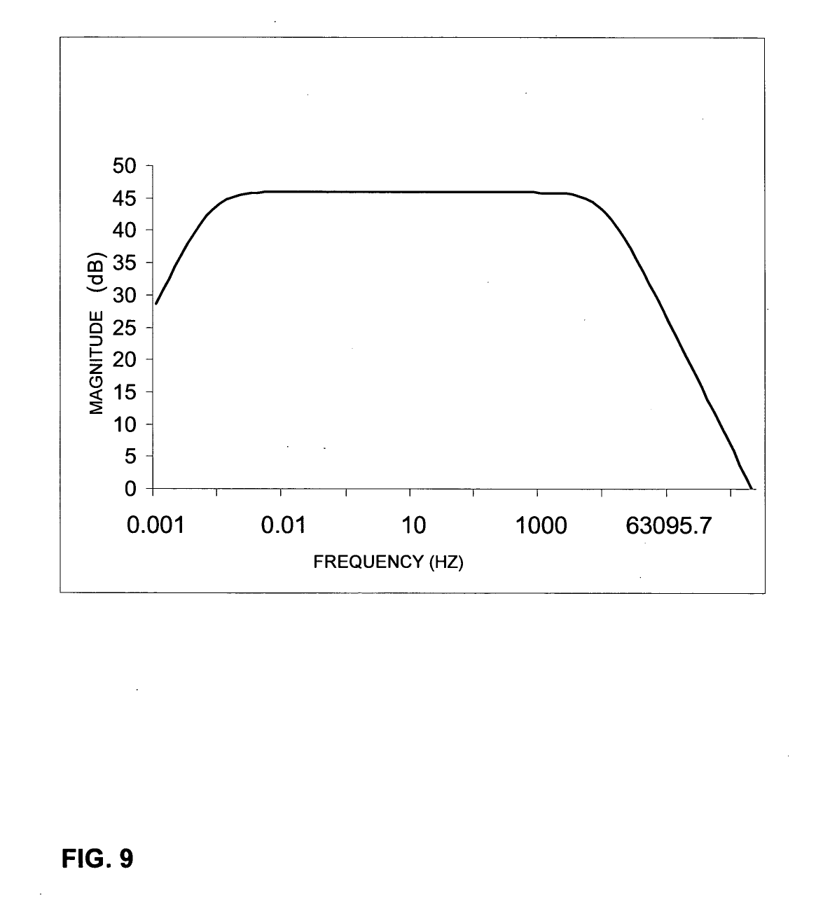

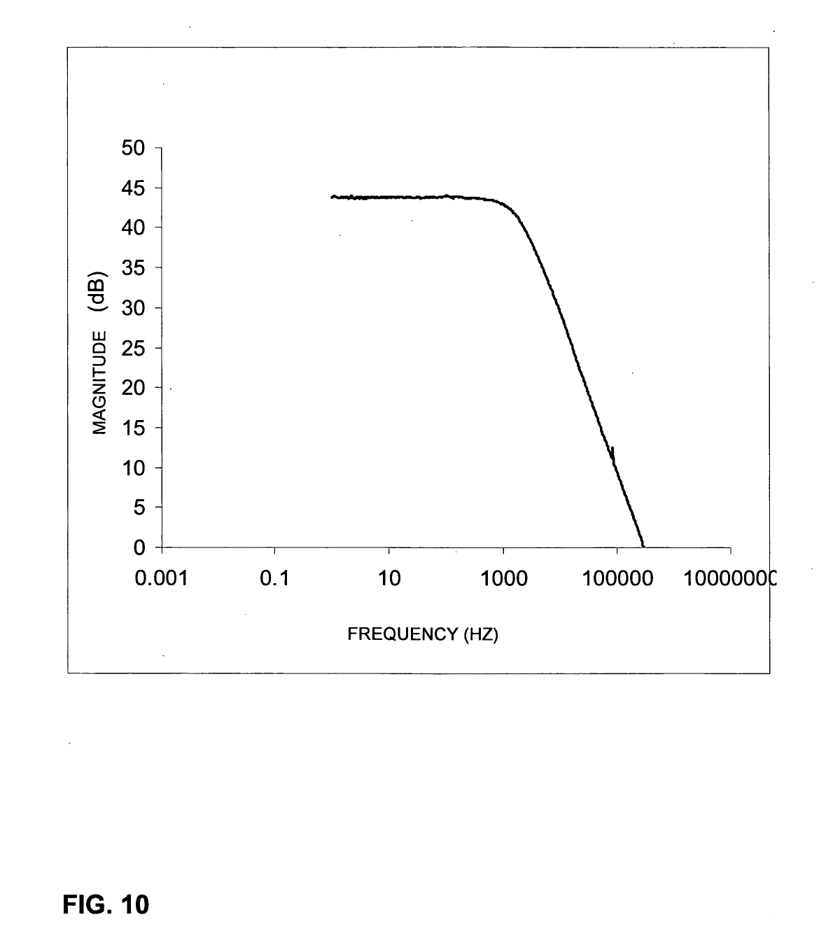

An example of circuit 30 has been fabricated. The fabricated circuit consumed approximately 0.07 square millimeters (mm²) area and 96 microWatts (μW) of power, respectively. The pre-fabricated simulated magnitude response is illustrated in FIG. 9 and closely matches the measured response illustrated in FIG. 10.

FIG. 6 is another circuit diagram illustrating feed-forward amplifier 12′ implemented as circuit 40 that includes low pass filter 22 implemented as a resistor-capacitor (RC) circuit comprising resistors 41, 42, and 45 and subtraction unit 24 implemented as an operational amplifier (op amp) 49 and resistors 46, 47. Resistors 41, 42 determine the cut-off frequency of low pass filter 22. The cutoff frequency of low pass filter 22 may be substantially equal to or less than the frequency component of the biological signal in order to generate the second and third signals from the biological signal as described previously. Importantly, the simulated gain of circuit 40 is approximately 26 dB at mid-band with a bandwidth of 0.010-1,000 Hz. In some embodiments, the ratio of the third signal to the biological signal is greater than approximately 100 dB.

The operation of circuit 40 can be explained as follows. Under DC conditions, capacitor 43 coupled to local ground 44 acts as an open circuit. Therefore, if the sum of resistors 41, 42 matches the resistance of resistor 45, the DC voltages at the inputs op amp 49 would match. As a result, the DC voltage at the output of feed-forward amplifier 12′ is cancelled. Under alternating current (AC) conditions, capacitor 43 provides finite impedance, which causes the AC signal voltage at the inputs of op amp 49 to have different voltages. Thus the inputs, i.e. the second signal and the biological signal, do not cancel. If op amp 49 is designed to provide gain, and the difference in voltage at the inputs of op amp 49 is amplified by the gain factor and appears at the output, i.e. the third signal.

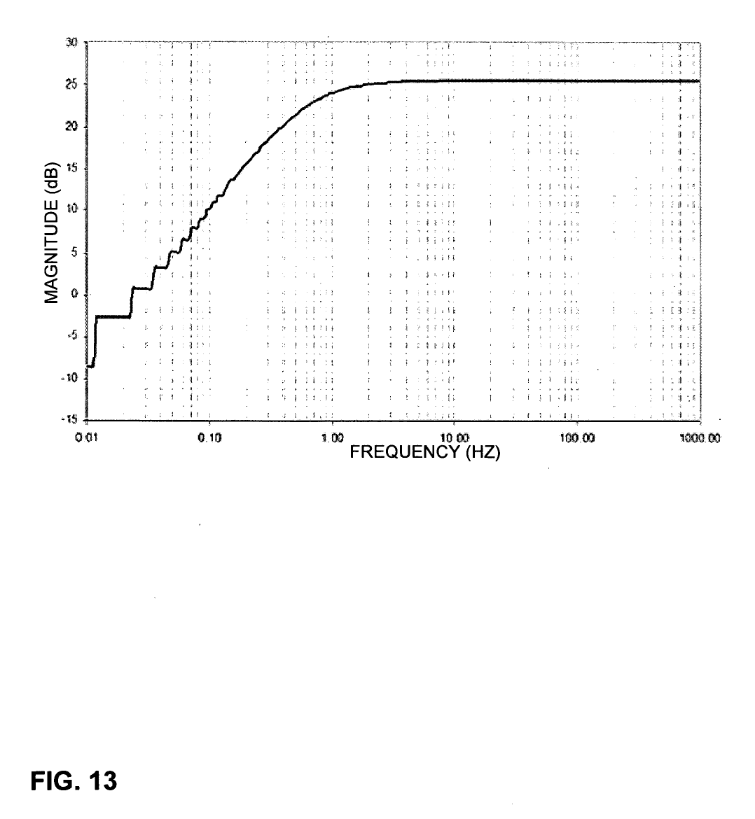

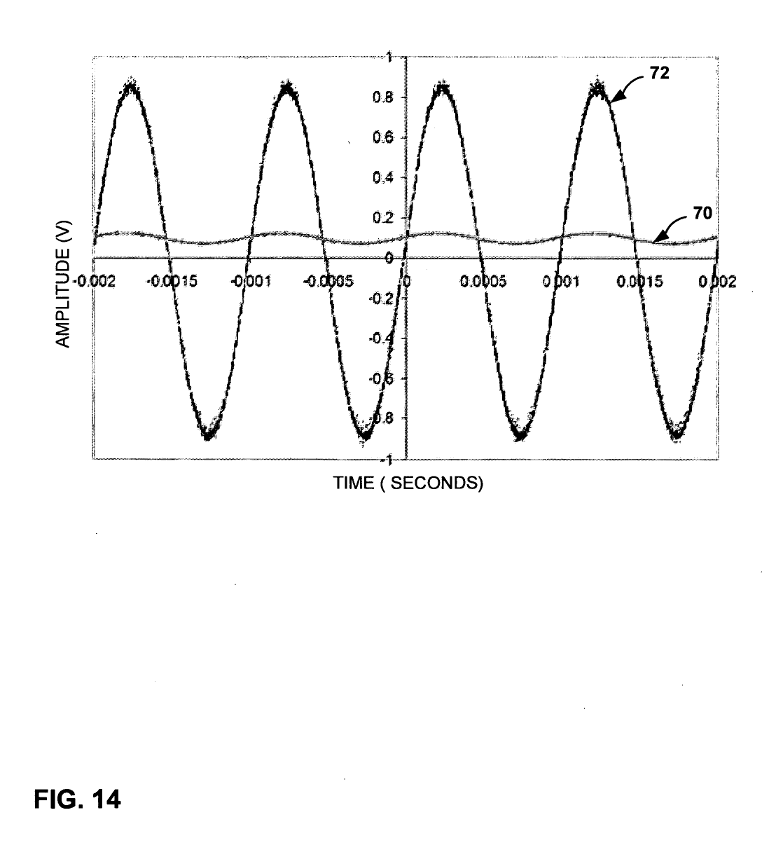

Circuit 40 may be particularly advantageous in monolithic implementations where it is difficult to control the precise value of the passives, i.e. resistors, across process and temperature but it is comparatively easy to control the relative matching to a great extent. In circuit 40, the relative ratios of resistors 41, 42, 45 and resistors 46, 47 are more important than the exact value of resistance at the input of the op-amp. In general, matching the values of the components is much easier than controlling the values themselves specifically. For example, circuit 40 may be designed such that the value of resistors 41, 42 are equal to 499 ohms, resistor 45 is equal to 998 ohms, and resistors 46, 47 are equal to 10,000 ohms. Capacitor 43 may then be designed to have a value of 1 mF. An example of circuit 40 was implemented with such values for testing on a PCB board. The results of the tests are illustrated in FIGS. 13, 14.

However, in an integrated design, resistors 41, 42 may have resistances of approximately 0.5×10¹⁴ ohms, resistor 45 may have a resistance of approximately 10¹⁴ ohms, and resistors 46, 47 may have resistances of approximately 1016 ohms. Such large integrated resistors may be implemented in CMOS using appropriately sized reverse-biased transistors, as illustrated in FIG. 5. Implementing large resistors in CMOS using reverse-biased transistors has the advantage of consuming less IC area. In such an integrated implementation, capacitor 43 may have a capacitive value approximately equal to or less than 0.2 pF.

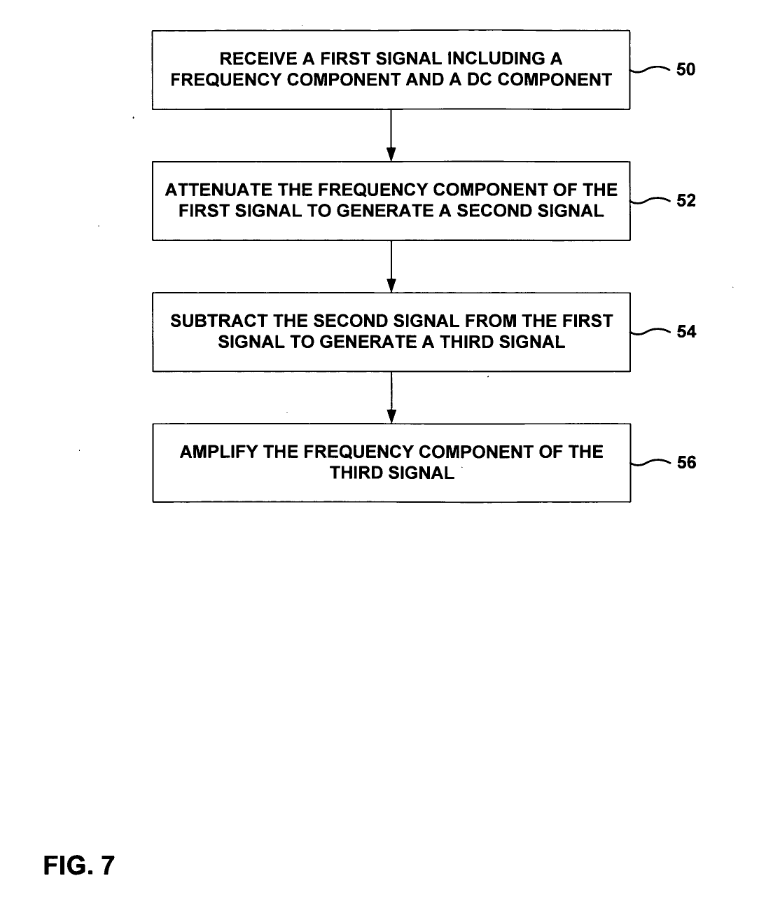

FIG. 7 is a flowchart illustrating an example method that may be implemented by a feed-forward amplifier 12 of FIG. 3. In general, feed-forward amplifier 12 receives a biological signal including a frequency component and a DC component (50). Low pass filter 22 receives the biological signal as an input and outputs a third signal. Feed-forward amplifier 12 provides a path that allows the biological signal to also be received as an input by subtraction unit 24. The amplitude and phase characteristics of the biological signal are not changed by the path provided by feed-forward amplifier 12. In any event, low pass filter 22 attenuates the frequency component of the biological signal to generate a second signal (52). The second signal is received as an input by subtraction unit 24. Subtraction unit 24 subtracts the second signal from the biological signal to generate a third signal (54). Thus, the input of low pass filter 22 is subtracted with the output of low pass filter 22. Because low pass filter 22 passes the DC component of the biological signal but attenuates the frequency component of the biological signal, the second signal includes primarily the DC component of the biological signal. Consequently, subtracting the second signal from the biological signal results in the third signal including primarily the frequency component of the biological signal. In some embodiments, the frequency component of the biological signal is less than approximately 7 Hz. Following subtraction of the input with the output of low pass filter 22, subtraction unit 24 may amplify the frequency component of the third signal (56).

In general, feed-forward amplifier 12 substantially cancels the DC offset of the biological signal and amplifies low frequency signals with a high DC offset rejection ratio. In some embodiments, the DC offset rejection ratio of the third signal relative to the biological signal may be greater than approximately 100 decibels. As described previously, amplifier 12 may be implemented with a low pass filter coupled to a subtraction unit, where the inputs of the subtraction unit are the input of the low pass filter and the output of the low pass filter. In general, the low pass filter passes the DC component of the biological signal while attenuating the frequency component of the biological signal in order to generate a second signal. Therefore, the second signal includes primarily the DC component of the biological signal. The subtraction unit subtracts the second signal from the biological signal. Subtracting the second signal with the biological signal causes the DC component of the biological signal to be substantially cancelled while the frequency component is not. As described previously, the subtraction unit may also be designed to amplify the third signal.

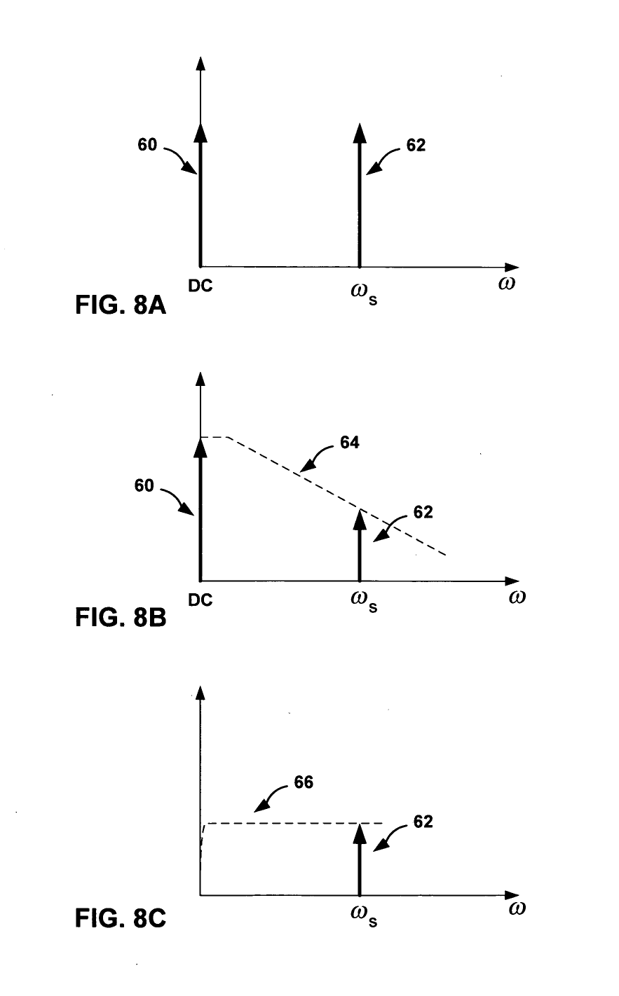

FIGS. 8A-8C are graphs illustrating the frequency spectrum representation of the DC component 60 and frequency component 62 of the biological signal, the second signal, and the third signal, respectively. In particular, frequency component 62 is illustrated as having frequency ωₛ. FIG. 8A illustrates DC component 60 and frequency component 62 of the biological signal. DC component 60 and frequency component 62 of the biological signal are illustrated as having equal magnitude. FIG. 8B illustrates the second signal as having primarily DC component 60 of the biological signal. In particular, DC component 60 is illustrated as having approximately twice the magnitude as frequency component 62, although the invention is not limited as such. Generally, a low pass filter, with magnitude response 64, reduces the magnitude of a frequency having twice the cutoff frequency of the filter by half. FIG. 8C illustrates the third signal having primarily frequency component 62 of the biological signal. In particular, DC component 60 is illustrated as having zero magnitude due to the subtraction of the second signal from the biological signal because of the magnitude response 66 of feed-forward amplifier 12.

FIG. 9 is a graph illustrating the simulated magnitude response of the amplifier 12′ illustrated in FIG. 5. The simulated magnitude response clearly illustrates a gain of 44 dB with a bandwidth of 0.05-6,000 Hz.

FIG. 10 is a graph illustrating the measured magnitude response of the amplifier illustrated in FIG. 5. It is important to note that measurements are taken from 1-1,000,000 Hz only. However, in accordance with the simulated response illustrated in FIG. 9, the measured response illustrates a gain of approximately 44 dB over a bandwidth of approximately 1-6,000 Hz.

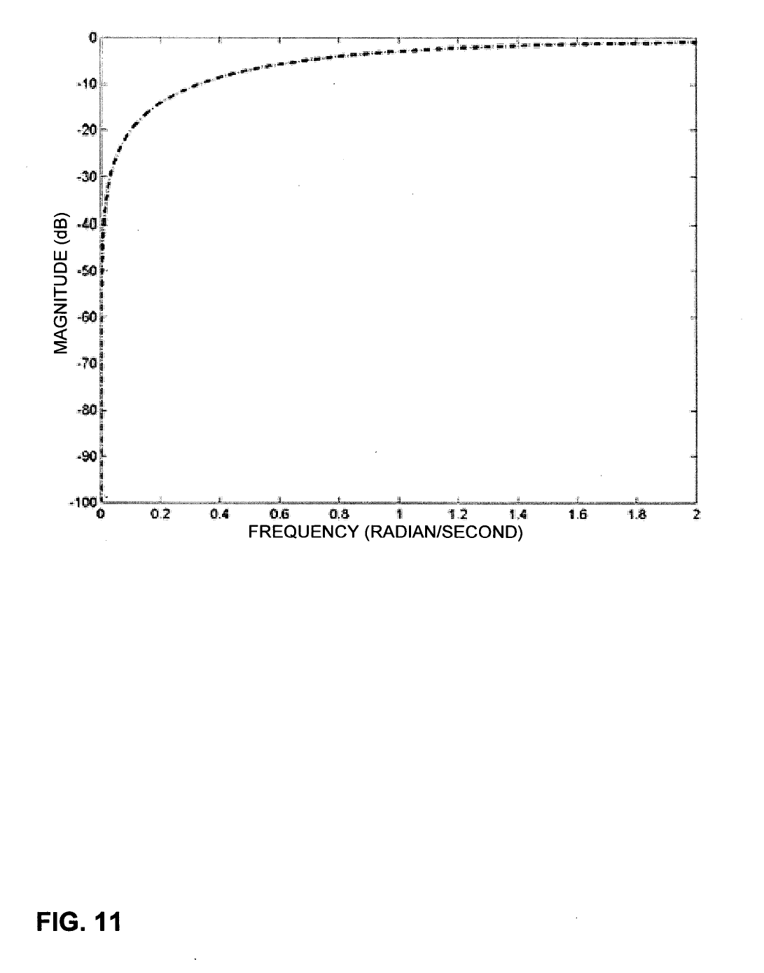

FIG. 11 is a graph illustrating the magnitude response of a feed-forward amplifier similar to that illustrated in FIG. 4 with amplification factor “G” equal to 1. The simulated magnitude response clearly indicates a very high quality factor (Q), −100 dB notch at DC. By increasing the gain of feed-forward amplifier 12″ in FIG. 6, the pass-band gain can also be increased correspondingly.

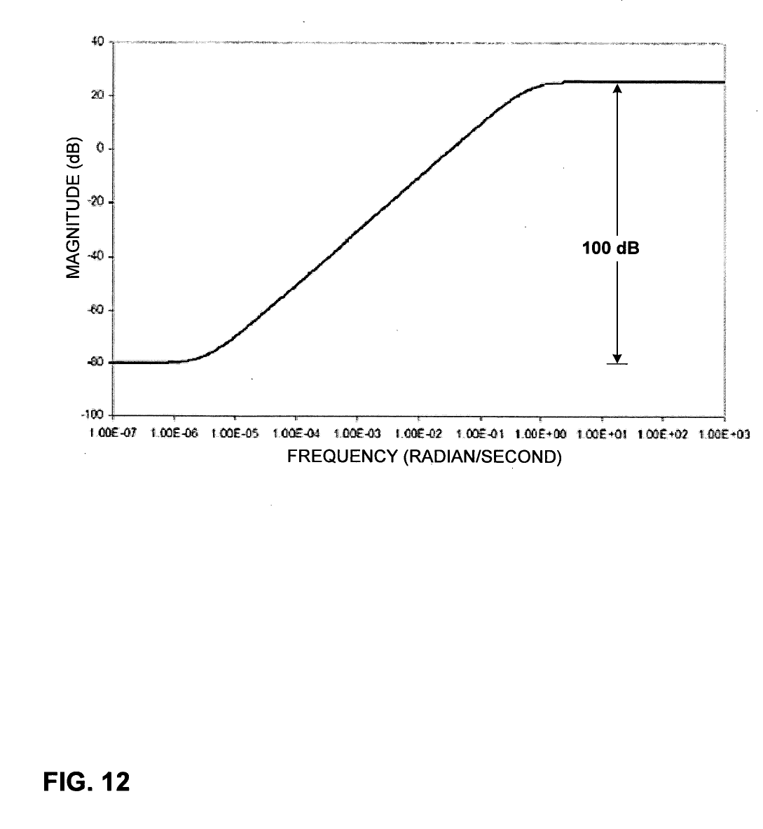

FIG. 12 is a graph illustrating the simulated frequency response of a feed-forward amplifier similar to that illustrated in FIG. 6 from 0.1 μHz to 1 kHz. The important result to note is that the ratio of the DC offset rejection to the gain in the pass-band is greater than approximately 100 dB. The DC cancellation is approximately 80 dB in this case because of the limitations of the commercial amplifier chosen, i.e. op amp 49. The DC offset rejection ratio may be increased to a value greater than 100 dB if the gain in the pass-band were increased.

A Keithley 619 Electrometer was used to measure the DC offset cancellation of circuit 40. Before measuring the DC offset cancellation, steps were taken to reduce the systemic DC offset of the op-amp itself by splitting the trimming potentiometer to smaller values. For a IV DC input, the output from circuit 40 was measured at approximately 450 μV, which equates to −80 dB suppression and matches the simulated results illustrated in FIG. 11.

FIG. 13 is a graph illustrating the measured frequency response of a feed-forward amplifier similar to amplifier 12″ illustrated in FIG. 6 from 10 mHz to 1 kHz. The frequency response was measured using AP Instruments' Model 200 Analog Network Analyzer (0.01 Hz-15 MHz). FIG. 13 has sharp steps at low frequencies because of the instruments limitation at such low frequencies. The measured result matches the simulated result in FIG. 12 closely. In particular, FIG. 13 illustrates approximately 26 dB gain in the pass-band and approximately −8 dB attenuation at 0.01 mHz. FIG. 13 illustrates a steady 26 dB gain at 6 Hz.

FIG. 14 is a graph illustrating the measured transient response of a feed-forward amplifier similar to amplifier 12″ illustrated in FIG. 6 at 1 kHz. Measurements were obtained using the Tektronix TDS 3012 digital phosphor oscilloscope. A Hewlett Packard 33120A function generator was used to feed circuit 40 a 100 mV peak-to-peak, 1 KHz sine input riding on a 100 mV DC-DC offset 70. The output of circuit 40 (72) is a 1.75 V peak-to-peak signal, at approximately 25 dB gain, with no visible DC-DC offset.

Various embodiments of the invention have been described. These and other embodiments are within the scope of the following claims.

1. A method comprising:
receiving a biological signal including a frequency component and a DC (direct current) component;
attenuating the frequency component of the biological signal to generate a second signal; and
subtracting the second signal from the biological signal to generate a third signal.

2. The method of claim 1, wherein the second signal includes primarily the DC component of the biological signal.

3. The method of claim 1, wherein the third signal includes primarily the frequency component of the biological signal.

4. The method of claim 3, wherein the third signal does not include the DC component.

5. The method of claim 1, wherein the DC component has an amplitude on the order of approximately 1000 times an amplitude of the frequency component.

6. The method of claim 1, wherein the third signal includes an amplification of the frequency component of the biological signal.

7. The method of claim 1, further comprising attenuating the frequency component to generate the second signal defined such that when the second signal is subtracted from the biological signal to generate the third signal the frequency component becomes amplified in the third signal.

8. The method of claim 1, wherein a transfer function of the third signal relative to the first signal is substantially given by the following equation where s is the frequency component of the biological signal, U(s) is the biological signal, Y(s) is the third signal, and G is an amplification of the frequency component of the third signal:
$$H ( s ) = \frac{Y ( s )}{U ( s )} = \frac{- Gs}{s + 1}$$

9. The method of claim 1, wherein an offset rejection ratio of the third signal relative to the biological signal is greater than approximately 100 decibels.

10. The method of claim 1, wherein attenuating the frequency component comprises substantially removing the frequency component.

11. The method of claim 1, wherein the frequency component of the biological signal is less than approximately 7 Hz.

12. An amplifier comprising:
a filter to receive a biological signal including a frequency component and a DC (direct current) component and to attenuate the frequency component to generate a second signal; and
a subtraction unit to subtract the second signal from the biological signal to generate a third signal.

13. The amplifier of claim 12, wherein the second signal includes primarily the DC component of the biological signal.

14. The amplifier of claim 12, wherein the third signal includes primarily the frequency component of the biological signal.

15. The amplifier of claim 12, wherein the DC component has an amplitude on the order of approximately 1000 times an amplitude of the frequency component.

16. The amplifier of claim 12, wherein the third signal includes an amplification of the frequency component.

17. The amplifier of claim 12, wherein the filter is defined such that when the second signal is subtracted from the biological signal to generate the third signal the frequency component becomes amplified in the third signal.

18. The amplifier of claim 12, wherein a transfer function of the amplifier is substantially given by the following equation where s is the frequency component of the biological signal, U(s) is the input to the filter, Y(s) is output of the filter, and G is an amplification of the frequency component of the third signal:
$$H ( s ) = \frac{Y ( s )}{U ( s )} = \frac{- Gs}{s + 1}$$

19. The amplifier of claim 12, wherein the third signal does not include the DC component.

20. The amplifier of claim 12, wherein an offset rejection ratio of the amplifier is greater than approximately 100 decibels.

21. The amplifier of claim 12, wherein the filter substantially removes the frequency component from the biological signal to generate the second signal.

22. The amplifier of claim 12, wherein the filter is a low pass filter.

23. The amplifier of claim 12, wherein the filter has a cutoff frequency less than approximately 7 Hz.

24. The amplifier of claim 12, wherein the filter and subtraction unit are fabricated on an integrated circuit.

25. The amplifier of claim 12, wherein the filter is a reverse-biased positive metal-oxide semiconductor (PMOS) transistor electrically coupled to a capacitor.

26. The amplifier of claim 25, wherein the capacitor has a value of less than approximately 0.2 pico Farads.

27. The amplifier of claim 12, wherein the subtraction unit is a low-noise complimentary metal-oxide semiconductor (CMOS) operational transconductance amplifier.

28. The amplifier of claim 12, wherein the amplifier has gain of approximately 44 decibels (dB).

29. An electro-physiological system comprising:
a set of electrodes to detect a set of biological signals; and
an amplifier comprising a filter to receive a selected biological signal including a frequency component and a DC (direct current) component and to attenuate the frequency component to generate a second signal, and a subtraction unit to subtract the second signal from the selected biological signal to generate a third signal.

30. The system of claim 29, further comprising an analog-to-digital converter to receive the third signal and convert the third signal to digital values.

31. The system of claim 29, further comprising a set of buffers associated with the set of electrodes and a multiplexer to select electrodes of the set of electrodes to identify the selected biological signal.

32. The system of claim 29, further comprising a common-mode noise subtractor to attenuate common-mode noise of the selected biological signal.

33. The system of claim 29, wherein the second signal includes primarily the DC component of the selected biological signal.

34. The system of claim 29, wherein the third signal includes primarily the frequency component of the selected biological signal.

35. The system of claim 29, wherein the DC component has an amplitude on the order of approximately 1000 times an amplitude of the frequency component.

36. The system of claim 29, wherein an offset rejection ratio of the amplifier is greater than approximately 100 decibels.

37. The system of claim 29, wherein the filter has a cutoff frequency less than approximately 7 Hz.

38. The system of claim 29, wherein the filter and subtraction unit are fabricated on an integrated circuit.

39. The system of claim 29, wherein the filter is a reverse-biased positive metal-oxide semiconductor (PMOS) transistor electrically coupled to a capacitor.

40. The amplifier of claim 39, wherein the capacitor has a value of less than approximately 0.2 pico Farads.

41. The system of claim 29, wherein the subtraction unit is a low-noise complimentary metal-oxide semiconductor (CMOS) operational transconductance amplifier.

42. An electro-physiological system comprising:
a set of electrodes to identify a set of biological signals, each of the biological signals including a frequency component and a DC (direct current) component; and
a set of amplifiers corresponding to the set of electrodes, wherein each amplifier of the set of amplifiers comprises a filter to receive one of the biological signals and to attenuate the frequency component of the received biological signal to generate a second signal, and a subtraction unit to subtract the second signal from the received biological signal to generate a third signal.

43. The system of claim 42, further comprising an analog-to-digital converter to receive the third signal and convert the third signal to digital values.

44. The system of claim 42, further comprising a multiplexer to select one of the biological signals and a common-mode noise subtractor to attenuate common-mode noise of the selected biological signal.

45. The system of claim 42, wherein the second signal includes primarily the DC component of the selected biological signal.

46. The system of claim 42, wherein the third signal includes primarily the frequency component of the biological signal and does not include the DC component of the selected biological signal.

47. The system of claim 42, wherein the DC component has an amplitude on the order of approximately 1000 times an amplitude of the frequency component.

48. The system of claim 42, wherein an offset rejection ratio of the amplifier is greater than approximately 100 decibels.

49. The system of claim 42, wherein the filter has a cutoff frequency less than approximately 7 Hz.

50. The system of claim 42, wherein the filter and subtraction unit are fabricated on an integrated circuit.

51. The system of claim 42, wherein the filter is a reverse-biased positive metal-oxide semiconductor (PMOS) transistor electrically coupled to a capacitor.

52. The amplifier of claim 51, wherein the capacitor has a value of less than approximately 0.2 pico Farads.

53. The system of claim 42, wherein the subtraction unit is a low-noise complimentary metal-oxide semiconductor (CMOS) operational transconductance amplifier.
